(19) United States
(12) Patent Application Publication
(10) Pub. No.: US 2022/0233339 A1
(43) Pub. Date: Jul. 28, 2022

(54) RLJ 1 Point Brace for Walking Cast

(71) Applicant: Ricky L Jones, Choctaw, OK (US)

(72) Inventor: Ricky L Jones, Choctaw, OK (US)

(21) Appl. No.: 17/158,342

(22) Filed: Jan. 26, 2021

(51) Int. Cl. A61F 5/01; A61F 5/30

(52) U.S. Cl. CPC A61F 5/0111; A61F 2005/0188; A61F 5/30

(57) ABSTRACT

Braces also known as spacers that protect the heel of a person wearing a weightbearing cast, with multiple exemplary embodiments such as with a single rear brace or two braces attached to at least one point on either side of the foot, with one brace below the heel and the other behind the heel with a clearance space between each brace and the cast, to redirect loads from the heel to points behind the ankle and forward of the arch.

### TECHNICAL FIELD

This disclosure relates to medical devices.

### BACKGROUND

The problem with loads on casts is that the flesh of the foot inside the cast experiences the same loads. The heel is of particular concern because it develops ulcers with minimal contact of the cast with any surface. Applicant has found no notable solutions to this problem in the prior art.

This inventor arrived at this design as an orthopedist, in a Technician's capacity, over the course of 30 plus years of experience. It is a necessary improvement to the art of cast making.

This disclosure describes an apparatus, and similar nonlimiting exemplary embodiments.

### SUMMARY

What is needed and not found in the prior art are devices that prevent heel sores in casts. This is done through braces that both redirect common forces and create awkward obstructions that discourage certain improper placements of the cast.

This disclosure provides a preferred brace and alternate embodiments. This specification also teaches the use of various embodiments. The methods can be used to install similar apparati that accomplish the same objectives. It is imperative to clarify that the braced walking cast can bear usual loading on the cast, but routine loading on the brace is generally limited to when a patient is in a sitting or lying down.

In this specification there are two loads discussed. The heel of the patient's foot should experience zero loading. The brace is designed to transfer any potential loading away from the heel of the foot. Thus, the brace will experience a second load and is rated accordingly. In sum, there is a heel load, and there is a brace load. This specification will always refer to the brace loading, and not the foot or heel loading unless specifically stated otherwise.

The intent of the brace herein disclosed is to redistribute the brace load away from the heel. In this specification, the preferred heel loading is zero pounds. However, to accommodate loads up to approximately 5 pounds, this bracing is helpful. BRACE LOADING OF 5 POUNDS IS IN NO WAY IMPLIED OR RECOMMENDED BY THIS METHOD, IT IS BEST THAT NO BRACE LOADING IS EVER PRESENTED. In this specification, 5 pounds is used as a symbolic, but approximately accurate load and does not imply a specific load limit. In practice, a practitioner would select from a variety of braces according to the patient's lifestyle and medical need.

In this specification, an embodiment of the brace has a maximum load rating of 25 pounds or even more for the expected durability of the brace during an expected load. The brace capability should exceed the safe loading for the patient. In such a nonlimiting embodiment, a practitioner should determine and warn the patient of the brace loading limits that are safe for the patient.

The solution herein disclosed redirects common axial forces on the cast from the heel area to points of contact above and forward of the heel, distributing point loads over the entire rear section of the cast. The primary embodiment includes one brace to protect the back of the heel from contacts. In an embodiment, the brace is secured to a finished leg cast. Securing is done by means that do not compromise the integrity of the finished cast by using nonlimiting examples like fiberglass rolls, plaster or other methods familiar to those in the field of orthopedics.

In a preferred embodiment, installing a geometrically rectangular brace to the cast behind the foot creates a normal and balanced horizontal position of the cast on a surface, with the toes straight up. A reason for the rectangular shape aligning the foot in this orientation is to prevent pivoting the cast along the leg center axis. This pivoting, in turn creates unwanted and unusual loading, which causes possible injury at the knee. In the case of a broken bone, such unwanted loading could affect the healing of the bone and alignment.

Further, a rectangular brace with 2 right angles that form corners connected by a flat section can be used to provide a stable, preferred loading effect. This encourages a patient to utilize normal, balanced preferred positioning. Further, geometry can be shaped according to a practitioner's preference to prevent unwanted loading. For example, a preferred load and shape could protect an incision that might be aggravated by certain loading. Such geometry includes but is not limited to shapes that have flat sections according to another preferred loading and pointed sections according to nonpreferred loading, such loads being applied by a patient to the cast in the common use and in their particular lifestyle.

In this specification the word brace is considered synonymous with other terms used herein, including “bracket” and “spacer”. In this specification the concept of a cast is used as an exemplary application. One of ordinary skill in the art would see that this method would apply to bracing other applications such as splints and nonweightbearing casts.

Common loading refers to loads that many cast wearers would apply to the heel in normal activities for an injured person. As a nonlimiting example, this includes a forward force directed from the heel area toward the toes during the frequent placement of the cast horizontally aligned and resting on a horizontal surface.

In the preferred embodiment, one brace is used on a single weightbearing leg cast. In a nonlimiting embodiment, the brace is attached at 4 points including two points on the left side and two on the right. In another nonlimiting embodiment a single attachment point is used on either side. In a third embodiment, integral tabs or ears are incorporated on the brace to further stabilize the brace when secured.

In the preferred embodiment, the multiple attachment points per side and tabs make it is possible to redirect common forces to any “best” part of the cast using eccentric torque loading instead of single mounting attachments. That is to say, with the single attachment per side, all forces are directed to each point of attachment. A patient-protecting part of the body to which a load can be directed is to the back of the ankle—along the Achilles.

In another nonlimiting embodiment, to redirect the load, the rear brace attaches with at least two points on either side of the ankle for at least 4 points. Thereby, with the brace the common forces impact the brace instead of the back of the cast, and usually create a torque instead of a direct impact. To be clear, a single, common force applied to the cast in a direction toward the heel, would in turn be applied “off center” to multiple eccentrically located attachments, to create a torque around the braced cast assembly.

In an embodiment having two attachment points on each side, of the ankle area the first attachment point is lower than the other—one toward the sole of the foot and the other higher in the ankle area—perhaps ½″ apart. The attachment points are also slightly (e.g. V) offset forward and rearward. With this 4-point mounting, any common load directed toward the back of the heel would be received by the multiple attachment points unevenly—eccentrically. This would cause a torque and apply the force at the back of the leg or on the sole of the foot. With an alternative positioning of the 4 mounting points a common force and resulting torque could be applied wherever is best.

An alternative nonlimiting embodiment will also prevent contact at the heel. The idea is to add an audible “click” at the brace to notify the wearer that a maximum load has been experienced by the cast. In an embodiment the clicking device should reset with no additional human intervention—spring loaded perhaps—so that it is repetitive, notifying the wearer of his/her overloading. The intent of the design is to be disruptive and attention-getting for the wearer, and to create notice and an incentive to avoid overloading the cast.

Another alternative embodiment helps children who have casts. Children are notorious for overloading the cast at the heel. It is important to reiterate that: LOADING OVER 5 POUNDS IS IN NO WAY IMPLIED ENCOURAGED OR RECOMMENDED BY THIS METHOD. The suggestion is to install decorative but functional attachments. In a nonlimiting embodiment, the design has reshaped braces that are decorative and aesthetically appealing, but also functional designs. As a nonlimiting example, floral shapes of suitable size, shape and material are be attached as part of the method. As another example, the braces themselves may be formed from the supplier with pair of ears shape and a tail shape. In another example, the reshaping elements are formed and attached to the braces at the factory, or the same elements can be attached to the braces by the practitioner.

Another embodiment continues to use electronics that would allow both notice that an overload has occurred and there is an aesthetic advantage for younger wearers. Again: LOADING OVER 5 POUNDS IS IN NO WAY IMPLIED, ENCOURAGED OR RECOMMENDED BY THIS METHOD.

In a nonlimiting embodiment, Electronics include switches mounted on the braces to activate upon a 5 # overload, with red LED indicators that flash for 5 seconds when an overload condition is momentarily present. Electronics can include a sonolert—an audible alarm to indicate an overload condition. The alarm can be a click, for subtlety. The alarm can be a garish siren.

In an embodiment, Other electronics include a molded pressure pad with at least one pressure sensor that respond to 5 # loading. Schmersal sms4-500-500 is an exemplary pressure mat with about 244 sensors. This would be overkill but offers the concept of a low-profile mat with multiple input sensors. In this application, only 4 to 6 sensors are needed in a minimal arrangement.

In an embodiment, in place of the braces, a single, rigid, molded cap is installed using the methods herein disclosed and used as a shield around the entire heel area. Again: LOADING OVER 5 POUNDS IS IN NO WAY IMPLIED, ENCOURAGED OR RECOMMENDED BY THIS METHOD. The cap would secure to the cast similar to the braces—at two opposite sides of the heel. The cap, however, could be closer to the cast than ¼″ previously outlined. In an embodiment, the caps have electronic switches and lighted outputs that mount and operate similar to those previously discussed.

Also, a seal around the edges of the cap will seal the cap to the surface of the cast. This seal can also be made of a rigid material sufficient to create a ring of contact between the cap and the cast where the redirected forces would be applied along a sizeable contact area.

A familiar and similar concept is used to protect a broken nose from contact (often used for basketball). In this exemplary concept, the mask redirects any forces away from the nose area and to other parts of the face. One of ordinary skill in the art would be familiar with materials appropriate to the application, including but not limited to plastics, carbon fiber and metal.

In an embodiment, a direct force testing system is employed and installed with each brace. This embodiment would be useful as a test mechanism or in applications that require the accurate measurement of the forces applied to a cast and in turn applied to the leg and foot of the patient. Force sensing pads (a nonlimiting example is a pair of pads electronically connected by a normally open switch) are used at force transfer contact points and molded onto the cast by applying an additional pad at the places where the load is more optimally applied. The concept of a broad, soft pad at the heel was rejected because it could result in undesired contact and a resulting ulcer. However, the top of the foot, the bottom sole of the foot and the back of the ankle are mentioned herein as good places to direct/redirect forces. At these locations, an applied force can be measured to protect the patient. An installer places the force pad in contact with the cast (or even inside the cast to the cast liner or to the foot/leg) at the best points for measuring redirected contact forces—and then secures to the completed cast as needed. Force pad readings are communicated using direct wiring or externally mounted wireless devices.

Certain nonlimiting embodiments are outlined herein, and one of ordinary skill in the art would be aware of modifications or alternatives that would be similar. One of ordinary skill in the art would also be aware of tools and hardware to aid in the installation process, and would use such items to brace, stabilize, locate, prepare, install, secure and similarly apply the methods herein disclosed.

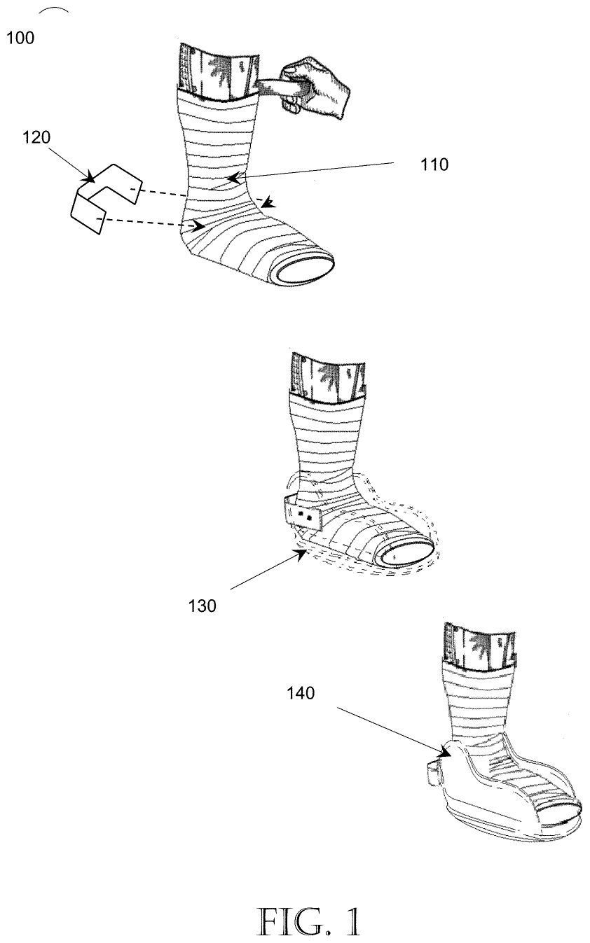

FIG. 1 is a nonlimiting demonstration of a cast, brace and walking shoe being applied to a right leg, typical of either leg

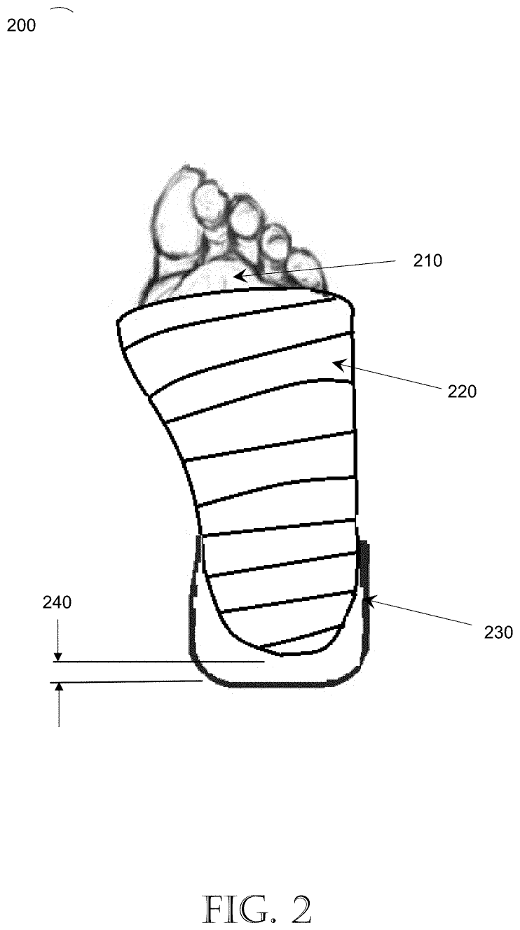

FIG. 2 is a nonlimiting demonstration of the bottom of a cast and having one brace, in a horizontal reclining position.

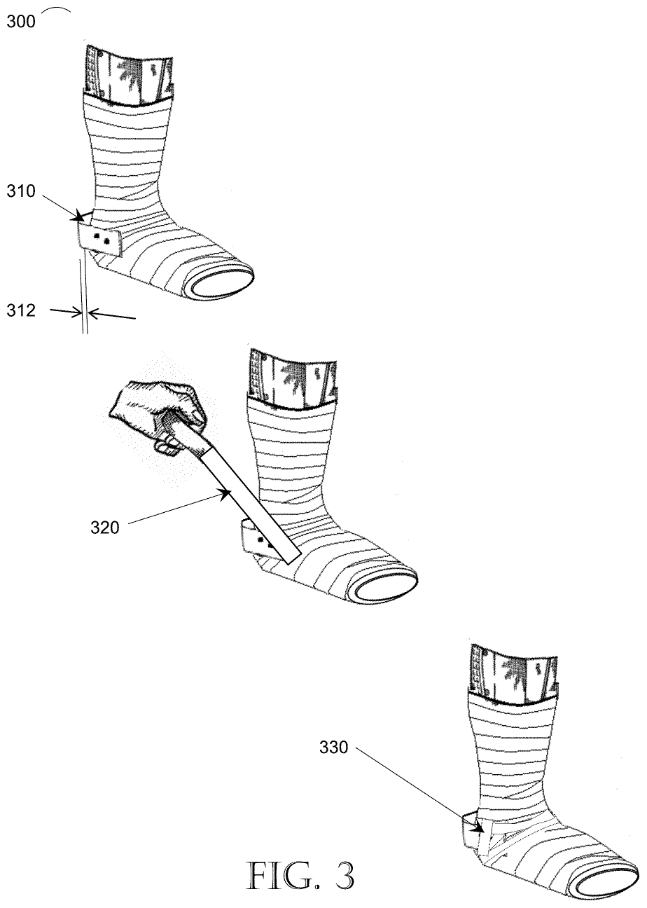

FIG. 3 is a nonlimiting demonstration of the brace during installation. Fiberglass or plaster is used to secure the brace to the cast.

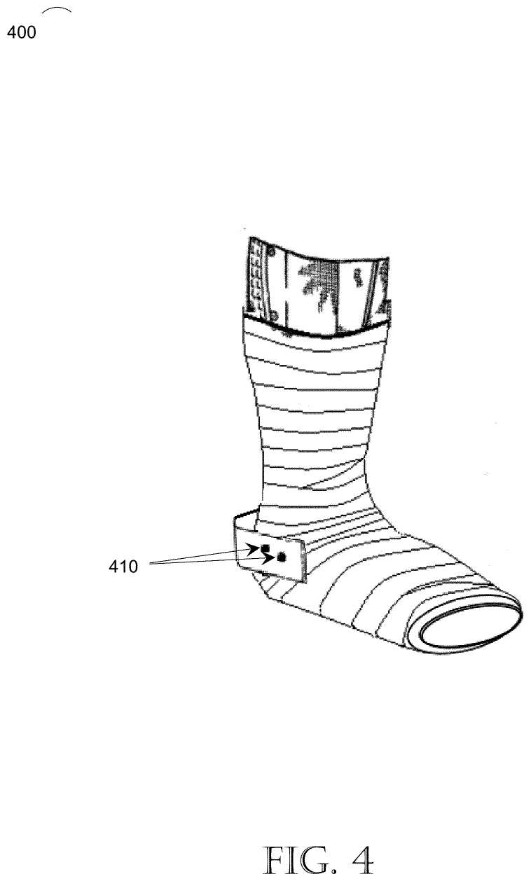

FIG. 4 is a nonlimiting demonstration of a two attachments per side brace. demonstrating the forces and resulting torques

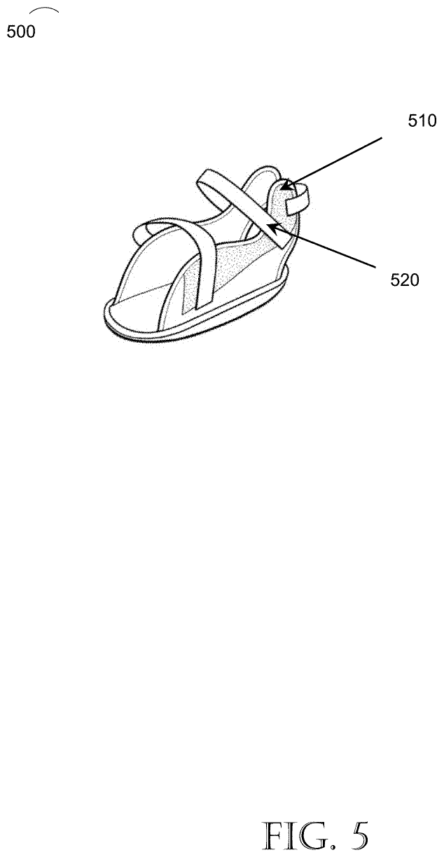

FIG. 5 is a nonlimiting demonstration of a type of cast shoe that may be applied to a walking/weight bearing cast

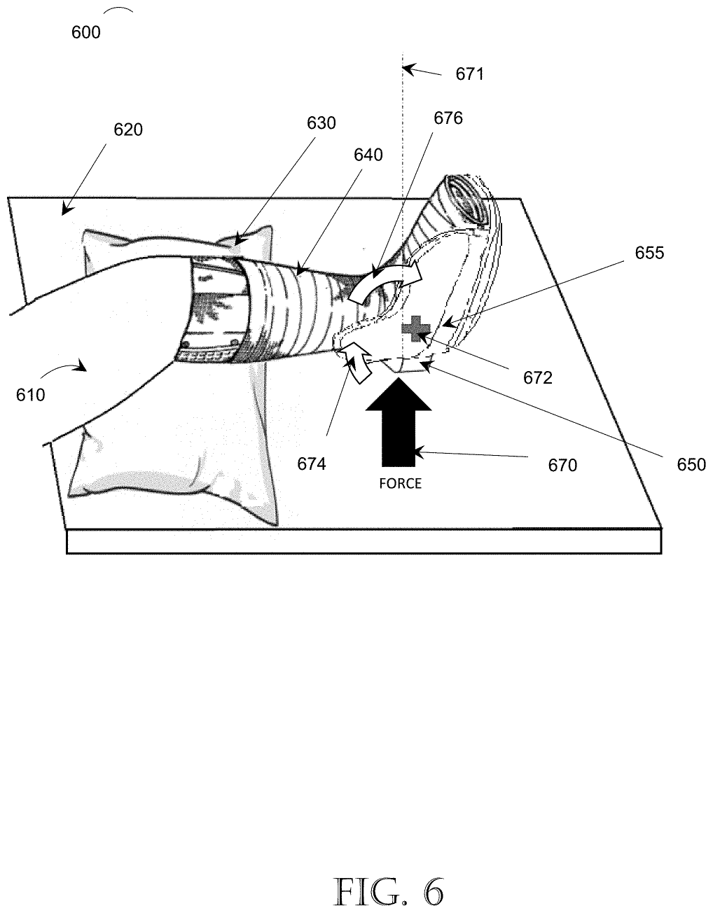

FIG. 6 is a nonlimiting demonstration of a cast with a brace, in a horizontal reclining position

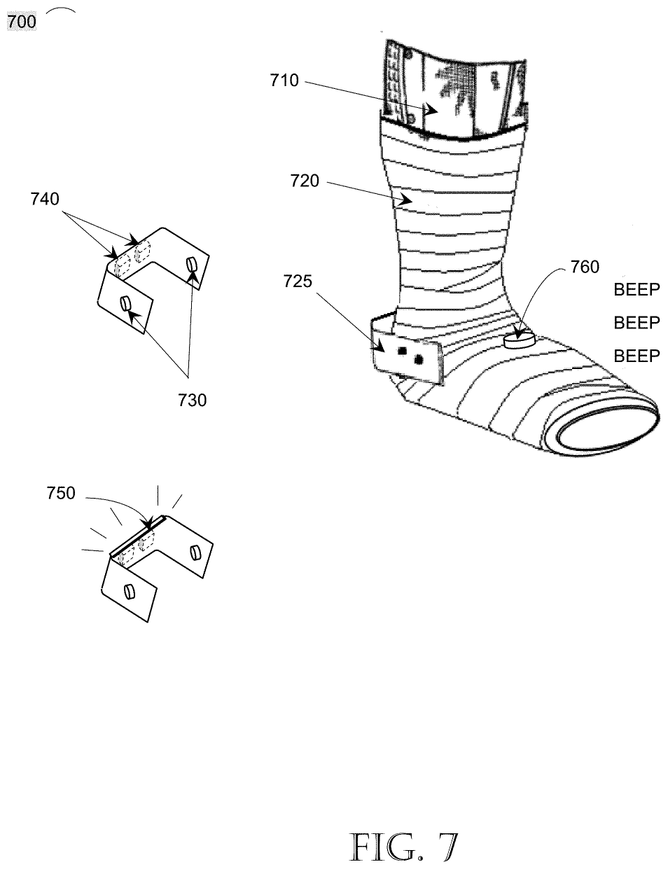

FIG. 7 is a nonlimiting demonstration of a cast and brace having electronic sonoalerts, sensors and mounting brace.

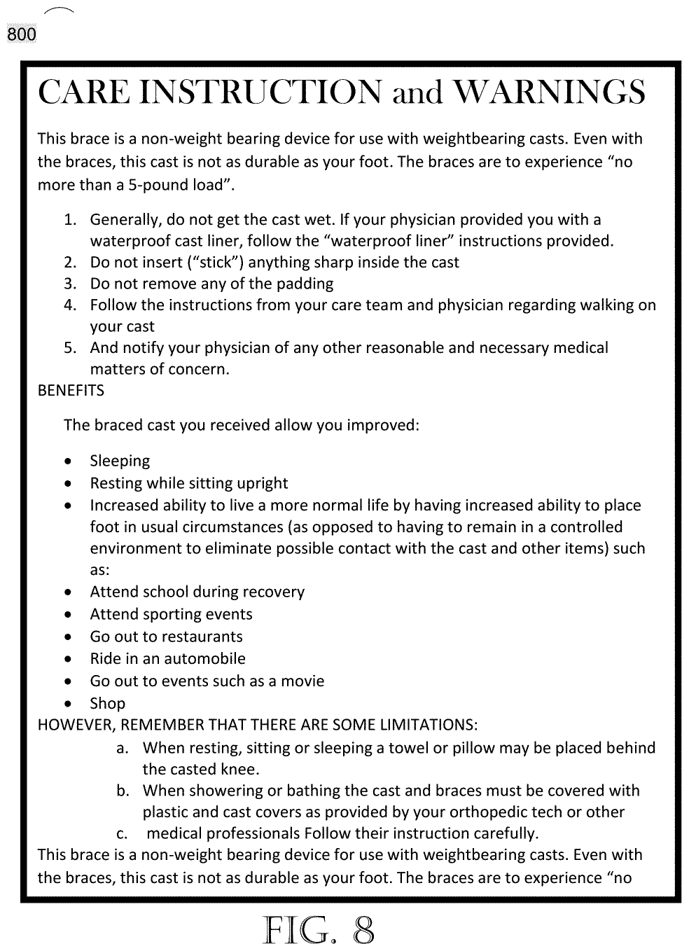

FIG. 8 is a nonlimiting demonstration of a warning label.

### DETAILED DESCRIPTION

FIG. 1 is a nonlimiting demonstration of a cast, brace and walking shoe being applied to a right leg, typical of either leg. 110 demonstrates the cast covering a right leg, with a demonstration of casting tape applied as part of constructing the cast. 120 demonstrates a typical brace, showing attachment areas on either side of the cast. 130 is demonstrative of any walking shoe of any typical design, with the shoe shown in phantom. 140 demonstrates an assembly view of any typical walking shoe, covering a cast with a typical brace. The 140 brace and other braces described herein can be attached or incorporated to the designs of any walking cast or boot of any typical design, using means familiar to those in the art. In this demonstration, the 140 brace is a single rear brace attached in two points at either side of the foot, forward of the heel as shown.

FIG. 2 is a nonlimiting demonstration of the bottom of a cast and having a brace, in a horizontal reclining position. 210 demonstrates foot and toes extending from a cast. 220 demonstrates the cast. 230 demonstrates the brace which is typically located and secured. 240 demonstrates necessary spacing between the brace and the cast, such spacing being adequate to avoid contact to the rear of the cast.

FIG. 3 is a nonlimiting demonstration of the brace during installation. A brace 310 is shown having a 312 spacing adequate to avoid contact between the brace and the rear of the cast. The 310 brace is shown with two attachment points. 320 demonstrates using fiberglass, plaster or other casting media to incorporate the 310 brace into the cast and cover the attachment points. 330 demonstrates an assembly view with the attachment point and edges covered with casting material.

FIG. 4 is a nonlimiting demonstration of a attachments per side brace. 410 attachment points are demonstrated offset in the fore aft and in the vertical axes. Such offset serves to create a torque when most forces are applied to the brace.

FIG. 5 is a nonlimiting demonstration of a typical walking boot to which a rear brace such as those described herein can be attached using other means that are obvious to those in the art. 510 is walking shoe that is installed over a cast to facilitate walking.

Drafting note: 520 demonstrates the securing straps and Velcro that are used to secure a walking shoe to a weightbearing cast. These securing straps are demonstrative, nonlimiting and are not shown in other views for clarity.

Note: though a plaster or fiberglass cast is demonstrated in the figures, other cast-like devices are used to therapeutically secure parts of the body. The brace concept will work with other cast-like devices like a post-operative cast shoe and a 3d printed cast.

FIG. 6 is a nonlimiting demonstration of a cast with a brace, in a horizontal reclining position demonstrating the forces and resulting torques. The patient 610 leg is shown on a 620 horizontal surface, such as a table or bed. The 630 pillow is demonstrative only of a typical suggestion from a practitioner, used to support the leg. 640 cast is shown with 650 brace assembly. In a 655 walking cast shoe, a single brace is used, and a bottom brace is not present. 670 demonstrates the force applied by the leg and cast resting on the table, shown pointing upward to demonstrate the effect on the cast and foot. Because the 650 brace is present, the 670 load is transferred along a 671 vertical axis to the 672 attachment point or points which are offset from the axis intersecting the patient's heel, creating a demonstrative 674 torque and 676 reactive torque applied to the cast and foot at points away from the patient's heel.

FIG. 7 is a nonlimiting demonstration of a cast and brace having electronic sonolerts and sensors. 710 demonstrates a liner inside the cast. 720 demonstrates the exterior of the cast. 725 demonstrates a cast brace. 730 demonstrates load sensors at the attachment points for the braces herein described to the cast. 740 demonstrates load sensors attached to the rear, exterior of the brace or flex sensors attached to the inside of the brace. 750 demonstrates lighting such as low voltage LED lamps or

LED lamp strips with a dispersion lens. 760 demonstrates a sonolert sound generator. The sensors, lamps and sonolerts are wired together so that the sensors activate the lamps and sonolerts when a preset load limit is experienced by the brace and sensors.

FIG. 8 is a nonlimiting demonstration of a warning label. The content of the label is fluid, but this label is included to demonstrate the necessity of warning labels and instructions for this design.

I claim:

1. At least one brace for casts attached to any protective cast over a patient's leg and foot, said cast positioned to absorb and redirect forces that would normally impact the heel of the cast and thereby impact the person's heel.

2. The at least one brace of claim 1 having tabs integral to the brace, such tabs located near attachment points and absorbing loads that could displace the brace while attached.

3. The brace of claim 1 having a single rear brace on weightbearing casts, having clearance to protect the heel from said impacts.

4. The brace of claim 1 as an assembly of at least two braces, at least located to the rear and to the bottom of a cast, having clearance between the cast and the brace to protect the heel from said impacts.

5. The at least one brace of claim 1, attached to at least one point on either side of the foot, with at least one brace behind the heel with a clearance space between the brace and the cast, the brace being attached offset from the axis of typical forces to redirect such typical forces away from the heel to points behind the ankle and forward of the arch.

6. The at least one brace of claim 1 having a maximum load rating of 5 pounds.

7. The at least one brace of claim 1 having a nonlimiting exemplary maximum load rating of 25 pounds, and practitioner safe loading recommendations to the patient according to the practitioner's expected load as to be expected by the patient's lifestyle.

8. The at least one brace of claim 1 having a geometry that is shaped according to a practitioner's preference to prevent unwanted loading, for example to protect an incision that might be aggravated by certain loading or to encourage a particular bone healing; such geometry including but not limited to shapes that have flat sections according to a preferred loading and pointed sections according to nonpreferred loading, such loads being applied by a patient to the cast in their particular lifestyle.

9. least one brace of claim 1 having a mechanical detail that produces a clicking sound when a force is applied, said force and corresponding detail being specified by a practitioner.

10. The at least one brace of claim 1 having decorative and aesthetically pleasing details that are integral to the brace or attached to the brace by the clinician, such as but not including nonlimiting examples of a, floral shapes of suitable size, shape and a pair of ears shape and a tail shape.

11. At least one brace for casts attached to any protective cast over a patient's leg and foot, said cast positioned to absorb and redirect forces that would normally impact the heel of the cast and thereby impact the person's heel; and having electronic sensors and circuitry mounted on the braces to activate when a maximum allowable load is applied to the sensors and brace.

12. The at least one brace and circuitry of claim 11 having at least one LED indicator to flash on and off, at least one time when an overload condition is sensed.

13. The at least one brace and circuitry of claim 11 having an electrical audible alarm to activate at least one sound when an overload condition is present.

14. The at least one brace and circuitry of claim 11 having at least one pressure pad having at least two pressure sensors that respond to a maximum load.

15. A rigid, molded cap attached to any protective cast over a patient's leg and foot, installed using attachment points on either side of the cast, having a spacing adequate to protect the cast from impacts especially in the heel area; the cap further having a seal around the edges to protect the surface of the cast from dirt and other intrusions.

16. The rigid, molded cap of claim 15 further having electronic sensors, lights, and audible alerts that activate with the application of a maximum force.
The rigid, molded cap of claim 15 further having sensors with data recording means to measure at least one force experienced during the wearing of a braced cast.
